(19) United States
(12) Patent Application Publication
(10) Pub. No.: US 2005/0100601 A1
(43) Pub. Date: May 12, 2005

(54) Virucidal activities of cetylpyridinium chloride

(75) Inventor: Charles L. Capps, Little Rock, AR (US)

Correspondence Address: VINSON & ELKINS L.L.P., 1001 FANNIN STREET, 2300 FIRST CITY TOWER, HOUSTON, TX, 77002-6760, US

(73) Assignee: ViraTox, L.L.C., Little Rock, AR (US)

(21) Appl. No.: 10/703,969

(22) Filed: Nov. 7, 2003

(51) Int. Cl. A61K 33/32; A61K 31/44; A61K 9/22

(52) U.S. Cl. 424/468; 514/358; 424/641

(57) ABSTRACT

This disclosure relates to inventive methods for inactivating viral pathogens in which the steps of the methods include: (a) providing a virucidal composition comprising a liquid media containing less than 1% weight per volume cetylpyridinium chloride; and (b) contacting the virucidal composition with a surface targeted for disinfection. This disclosure further relates to inventive virucidal compositions, in which the compositions include: (a) a liquid media; (b) cetylpyridinium chloride in solution in the liquid media at a concentration of less than 1% weight per volume; (c) an extender; and (d) an enhancer.

### REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to the use of a quaternary ammonium compound as a virucidal agent for surface contact disinfection and for the medical treatment and prevention of infection caused by viral particles. Specifically, the present invention relates to the use of cetylpyridinium chloride in low concentration solution for contact destruction of viral particles in a relatively short time period with subsequent residual associated toxicity to viral particles. More specifically, the present invention describes the composition and application of cetylpyridinium chloride from a dry composition powder to a solution for surface disinfection and as a spray or aerosol inhalant for respiratory treatment for the destruction or inhibition of the corona virus.

2. Description of the Related Art

Viruses are considered to be the smallest infectious agents capable of replicating in living cells. The precise mechanism of nucleic acid transfer to the host cell and the subsequent function of the host to promote production of additional progeny is not fully understood. Viruses have a core of nucleic acid surrounded by a protein coat or coats and may be furthered enveloped as in the case of the corona virus. The nucleic acids are of two types, ribose (RNA) or deoxyribose (DNA). This genetic material is responsible for directing replication once a suitable host is invaded. Without a protein coat, the nucleic acid is normally incapable of entering a cell. In general, the DNA viruses multiply in the nucleus of the host cell and RNA viruses in the cytoplasm. Some RNA viruses appear to emerge as buds from the cell membrane. Generally, the mixoviruses and rhinoviruses such as the corona virus are of the RNA types, while viruses such as the papovaviruses and the adenoviruses (herpes and pox) are DNA based. Corona viruses, such as those that cause the common cold, are known to infect a wide variety of hosts. For example, corona viruses are known to infect humans, dogs, cats, pigs and birds.

Virucides play a critical role in limiting or irradicating the deleterious presence of pathogenic viruses in a wide variety of settings, such as in hospitals, patient rooms, treatment facilities, and other surfaces in which the inactivation of viruses would have beneficial effects. Nevertheless, the need for potent and effective virucides must be counterbalanced against concerns for environmental safety.

Elimination of viral pathogens, especially those existing upon inanimate surfaces, where such pathogens may remain active for extended periods of time, has been a long standing challenge to maintaining an antiseptic environment in a wide variety of settings. A wide variety of disinfectants have been developed in attempts to provide an adequate mechanism for the elimination of such statically existing viral pathogens. However, many of these disinfectants tend to have less desirable characteristics. For example, some may be corrosive while others may be repugnant or cause discoloration of a treated area.

Cetylpyridinium chloride (CPC) is a quaternary ammonium compound as described in The Merck Index (The Merck Index, 11th Ed.; Merck & Co., Rahway, N.J. (1989); p. 311) with antiseptic and preservative properties. It is a cationic compound that is presently known to have broad based antibacterial properties, thus making it an active ingredient for the inactivation of both gram negative and gram positive bacteria. Examples of bacteria that are known to be susceptible are: Escherichia coli, Salmonella typhimurium, Pseudomonas aeruginosa, Bacillus anthracis, Bacillus subtilis, Campylobacter, Listeria, Staphylococcus aureus, and Streptococcus pneumoniae. Additionally, cetylpyridinium chloride is also known for its antifungal properties affecting such fungi as Candida albicans and spp., Saccharomyces cerevisiae, Torulopsis glabrata, Trichophyton sp., Aspergillus flavus and niger, Stachybotrys atra, Chaetomium globosum, Histoplasma capsulatum, Penicillium cyclopium, and Cladosporum resinae (Reed, R. H., “Cetyl Pyridinium Chloride: Status Report.” University of Northumbria School of Applied and Molecular Biology, January 2002).

The recognized broad spectrum antimicrobial antiseptic effect of CPC without disturbance of intra-oral bacterial flora has resulted in its common use in oral rinses and lozenges. Commercial oral rinse products include Scope® (Procter and Gamble), Cepacol® (J. B. Williams), and Act® (Johnson & Johnson). Formulation concentrations of CPC in these products range in the 0.04% to 0.05% and are generally recognized as safe levels while possessing the ability to be effective against undesired microbes. There are also multiple citations of CPC efficacy as an active ingredient in the reduction and inhibition of plaque and gingivitis, thus encouraging use as a dental hygiene constituent (Hunter-Rinderele et al., J. Clin. Res. 72:107113, 1997). However, much evidence exists wherein the efficacy of CPC as an antimicrobial is severely hindered by commonly used ingredients in the formulations of rinses, lozenges, dentifrices and oral care products (Addy et al., J. Dent. Res. 72:719, 1993). Other commercial antiseptic product applications include feminine washes and topical antiseptics for abrasions and minor wounds.

### SUMMARY OF THE INVENTION

The present invention includes compositions and methods for inactivating viruses residing on inanimate objects or within the respiratory tract of humans or other animals, especially the oral or nasal cavities. The present invention also includes methods and compositions for decontaminating areas, tools or devices infected by viral pathogens such as those commonly found in hospitals, public restrooms, transportation vehicles, and institutions where mass population would require sanitization of surfaces with maximum residual results.

The present invention includes the use of solubilized CPC in a liquid or other suitable media. Certain embodiments comprise solubilizing CPC in either potable or sterile water for use as a surface contact disinfectant or as a spray in oropharyngeal applications for the destruction of viral agents in the respiratory tracts of humans or other animals. While, still other embodiments of the present invention provide a virucidal kit comprising a CPC containing compound. In such kits, the CPC containing compound may be provided in a concentrated form for dilution to a working concentration prior to use.

Certain other embodiments of the present invention provide compositions that may be safely ingested by humans and other animals, thereby making the compositions and methods of the present invention suitable for treating humans and other animals, exposed to pathogenic viruses. In alternative embodiments, effective amounts of the CPC may be applied to the human and other animal being treated prior to exposure to the pathogenic viruses. Therefore, the present invention includes compositions and methods for both the prevention and treatment of viral infections. In these particular embodiments, the present invention may comprise an inactivating composition suitable for pharmaceutical administration to humans and/or other animals. Such compositions suitable for pharmaceutical administration may also comprise one or more pharmaceutically acceptable carriers. These compositions may be applied topically to mucus membranes or oral surfaces including the buccal cavity and throat as a spray or oral rinse (or other acceptable form) or to any other suitable surface of the body, including dermal surfaces and open wounds, to treat or prevent viral infections.

### Definitions

“Inactivate” or “Inactivating,” means having the ability to destroy, eliminate or reduce the capacity of a viral pathogen to infect and/or cause a pathological response in a host; wherein such inactivating ability preferably provides at least a one-log order reduction (90%) in the viral pathogens ability to infect and/or cause a pathological response in a host, more preferably a two-log order reduction (99%), and most preferably a three-log order reduction (99.9/o).

“Contact” or “contacted” refers to bringing one or more of the compositions of the present invention into contact with a viral pathogen such that the composition(s) of the present invention may inactivate the viral pathogens.

“Enhancers” describes compounds or substances that act to enhance or accelerate the ability of CPC to inactivate viral pathogens.

“Extenders” describes compounds or substances that act to extend or prolong the inactivating ability of CPC on viral pathogens.

“Pharmaceutically acceptable” refers to compositions that do not produce significant adverse reactions when administered or applied to a particular host (e.g., a human or other animal).

“Pharmaceutically acceptable carrier” includes any and all solvents, media, coating or wetting agents, and the like, as well as certain enhancers and extenders, which when utilized remain pharmaceutically acceptable.

### DETAILED DESCRIPTION OF SPECIFIC EMBODIMENTS

The present invention provides for compositions of solubilized CPC in a liquid or other suitable media and the use of these compositions to inactive viral pathogens. The present invention further comprises solubilizing CPC in any suitable liquid media, such as potable or sterile water or an isotonic saline solution, for uses as a surface contact disinfectant or as a spray in applications for the destruction of viral agents in the respiratory tracts of humans or other animals. Solubilizing CPC should be done by adding selected amounts of CPC powder into the liquid media choice. Alternative embodiments of the compositions of the present invention also include the addition of a stabilizing agent, such as polypropylene glycol, to stabilize and reduce the freezing point of the resulting solution.

1. Surface Contact Disinfectant

In embodiments of the present invention, which are directed toward surface contact disinfectants and disinfection, the CPC solution concentrations may range from greater than 0.0% up to around 1.0%, the concentrations varying as to specific use. In certain embodiments directed toward household applications, a solution concentration of greater than 0.0% to 1.0%, preferably from 0.01% to 0.5%, more preferably from 0.02% to 0.05%, most preferably around 0.025%, would provide a benign but effective antibacterial disinfectant that would additionally promote the inactivation of certain viral agents, including corona virus. In applications requiring more aggressive disinfection where more virulent strains of viral organisms exist, higher concentrations of CPC up to around 1.0% may be required to assure maximal contact destruction of contaminants on surfaces with residual presence to inhibit further contamination within a reasonable period of time.

CPC also possesses a surfactant property and aggressively adheres to solid surfaces thus promoting a residual action. Extenders, such as cellulose ethers, including but not limited to hydroxypropyl methylcellulose, or sodium carboxymethylcellulose, may be used in various concentrations as adjuncts in certain embodiments of the present invention for extending or enhancing the toxicity and longevity of residual inactivating effect of CPC. These compounds are widely recognized as viscosity modifiers, emulsifiers, protective colloids in emulsions, and as film forming agents. Viscosity properties are dependent on the degree of substitution as a water soluble polymer. For example, in the case of hydroxypropyl methylcellulose, a substitution of 0.01% to 2.5% will cause film-forming and encapsulation, while 0.5% to 5.0% of carboxymethylcellulose will accomplish similar results. In certain embodiments of the present invention these extender compounds may increase surface attachment and upon drying may result in a solid film with varying degrees of water resistance. In certain other embodiments, it is contemplated that the extender is a non-ionic detergent. Non-ionic detergents that find use in the present invention include, but are not limited to varying concentrations, which would be readily ascertainably to one of skill in the art, of Tween 80, Triton, Tyloxapol, Pluronic and Span.

Other embodiments of the present invention may comprise the addition of enhancers which function to accelerate the inactivating properties of CPC. Alternative embodiments of the compositions of the present invention may comprise solutions containing zinc or copper compounds, which include but are not limited to zinc oxide, zinc acetate, zinc bacitracin, zinc carbonate, zinc citrate, zinc iodate, zinc iodide, zinc peroxide, zinc propionate, zinc stearate, zinc salicylate, cuprous iodide, and cupric oleate. The concentrations necessary will vary from enhancer to enhancer but would be readily ascertainable to one of skill in the art. For example zinc oxide may be used as an enhancer at concentrations of greater than 0.0% weight per volume to as high as around 2.5% weight per volume.

The compositions and methods of the present invention also contemplate combining the CPC compositions with various cleansing applicators, such as wipes, mop head, sprays, and the like.

The methods of the present invention comprise contacting CPC containing compositions for a time sufficient to inactivate the pathogenic agent or to inhibit the growth of the agent. Contact times sufficient for the inactivation of pathogenic agents may vary from agent to agent but will be readily ascertainable to one of skill in the art. Time periods will preferably range from greater than 0 seconds to around 3 minutes, more preferably from 30 seconds to 1 minute, most preferably a sufficient time period would be around 45 seconds. In other embodiments, the present invention provides a method of decontaminating an environmental surface harboring harmful or undesired pathogens. In one such embodiment, the pathogenic agent is associated with an environmental surface and the method comprises contacting the environmental surface with an amount of the composition sufficient for decontaminating the surface. While it may be so desired, decontamination need not result in total elimination of the pathogen, preferably decontamination will result in a least a one-log reduction (90%) in the presence of active viral pathogens, more preferably a two-log reduction (99%), most preferably a three-log reduction (99.9%). In some embodiments, the compositions and methods further comprise dyes, paints, and other marking and identification compounds as to ensure that a treated surface has been sufficiently treated with the compositions of the present invention.

The present invention further provides methods for protecting (e.g., protecting from contamination of a microorganism) or decontaminating an area (e.g., decontaminating an area by inactivating viral particles in the area) comprising exposing the area to a CPC containing composition comprising an extender and/or enhancer. The exposure times for such areas may vary from area to area and from extender and/or enhancer to extender and/or enhancer but will be readily ascertainably one of skill in the art. Such exposure times will preferably range from greater than 0 seconds to around 3 minutes, more preferably from 30 seconds to 1 minute, most preferably a sufficient time period would be around 45 seconds. These methods may be applied to any type of area or item (e.g., a medical device or treatment table or paper/cloth diapers or mats). For example, in some embodiments, the area may be any variety of hard or soft surfaces, whether porous or non-porous.

Other embodiments of the present invention provide a virucidal kit comprising a CPC containing compound. In such kits, the CPC containing compound may be provided in a concentrated form for dilution to a working concentration prior to use. These kits may also contain one or more extenders and or enhancers. Furthermore, these kits may provide instructions as to a method of use and or desired dilution ratios to be employed. In some embodiments such kits may provide methodologies for targeting particular types of viral pathogens, which may comprise variations in CPC concentrations, or the use of particular extenders and/or enhancers.

2. Pharmaceutical Applications

The present invention also comprises compositions and methods that are useful in the treatment and prevention of virally mediated pathogenic responses in humans and other animals. Viruses mediating such responses would preferably be viruses infecting the respiratory tract of humans and other animals, especially the upper respiratory tract. For example, suitable CPC containing compounds are effective in inactivating the corona virus, which is capable of infecting a wide variety of hosts including humans, dogs, cats, pigs and birds. The compositions and methods of the present invention will also be useful in the treatment and prevention of virally mediated pathogenic responses on dermal surfaces and open wounds in humans and other animals. For example, in the treatment and prevention of pathogenic states which result from dermal contact with antrax.

In some embodiments, the present invention provides compositions and methods suitable for treating humans and other animals, exposed to pathogen viruses. In alternative embodiments, effective amounts of the CPC are applied to the human and other animal being treated prior to exposure to the viral pathogens. Therefore, the present invention comprises compositions and methods for both the prevention and treatment of viral infections. In these particular embodiments, the present invention may comprise an inactivating composition suitable for pharmaceutical administration to humans and/or other animals.

In the case of upper respiratory tract infections, such as the common cold, the viral infection remains primarily located in the throat and respiratory tract and causes a susceptibility to infection by other microbial organisms by weakening tissues and inviting secondary bacterial infections. In the present invention, delivery of CPC to these areas via a spray, such as by a pump device or a propelled aerosol, would enable contact destruction of the virus as well as secondary infectious bacterial agents sensitive to CPC. Such devices are readily available in the marketplace and include metered dose inhalants by Cambridge Consultants or Wilden. Pharmaceutically acceptable CPC containing compositions may be utilized in methods of the present invention to directly target tissue areas of infection within the host. For example, an oropharyngeal spray or rinse may by used to deliver suitable CPC containing composition to the throat, buccal cavity and/or nasal passages of an infected individual in concentrations which would be effective in inactivating the viral pathogens. Furthermore, such a spray or rinse may even be used to inactive viral pathogens upon suspected exposure prior to actual infection.

In the case of an oropharyngeal application, CPC in the range of greater than 0.0% to 0.5%, preferably 0.01% to 0.1%, more preferably 0.025% to 0.05%, in sterile solution would be applied as a spray to the area of the buccal cavity and throat to enable contact destruction of viral agents of the mucosal surfaces to reduce or eliminate infection of viral agents. The compositions and methods of the present invention are also useful in the treatment and prevention of virally mediated pathogenic responses in the lungs of humans and other animals. In the case of the treatment and prevention of viral pathogens in the lungs of humans and other animals, CPC containing pharmaceutically acceptable compositions may be applied as a mist or mist-like inhalant for direct contact with the tissues of the bronchial tract and lung alveoli and surrounding tissue. In such intra-lung applications, useful CPC concentrations in sterile solution may be in the range of greater than 0.0% to 0.5% weight per volume, preferably 0.01% to 0.1%, more preferably 0.025% to 0.05%. Additional affectation of viral infection may occur as the transport of the CPC solution ensues within cellular structures of tissue hosting viral agents. Since CPC is highly cationic, it is readily adsorbed and retained on oral and mucosal surfaces, providing a mechanism for extended residual action.

Such compositions suitable for pharmaceutical administration may also comprise one or more pharmaceutically acceptable carriers. These compositions can be applied topically to mucous membranes or oral surfaces including the buccal cavity and throat as a spray or oral rinse (or other acceptable form) or to any other suitable surface of the body, including dermal surfaces and open wounds, to treat or prevent viral infections.

Extenders may be used in compositions of the present invention in addition to or as pharmaceutically acceptable carries. Such extenders include but are not limited to the cellulose ethers hydroxypropyl methylcellulose or sodium carboxymethylcellulose which may be used in various concentrations. In certain other embodiments, it is contemplated that the extender is a non-ionic detergent. Non-ionic detergents that find use in the present invention include, but are not limited to varying concentrations, which would be readily ascertainably to one of skill in the art, of Tween 80, Triton, Tyloxapol, Pluronic and Span. Alternate embodiments of the present invention may contain suitable enhancers to accelerate the inactivating properties of CPC. For example, zinc, in the form of zinc oxide, may be included in the CPC containing compositions to accelerate the CPC mediated inactivation of viral particles. In still other embodiments of the present invention CPC containing compositions may contain both extenders and enhancers.

Additionally, in still other embodiments of the present invention, the formulations further comprise any number or variation of coloring or flavoring agents (e.g., dyes and peppermint oil).

While the invention has been described with particular reference to specific embodiments thereof, it is to be understood that the forms of the invention shown and described in detail are to be taken as preferred embodiments thereof. Various changes and modifications may be resorted to without departing from the spirit and scope of the invention as defined by the claims.

### EXAMPLE

The present invention derives from in vivo trials and challenge studies (1) in determining whether CPC may be effective as an antiviral agent and, if effective, the minimum inhibitory dosage; (2) in determining any adverse effects of the host cells and dosages thereto; and, (3) in comparing antiviral effects of CPC, benzalkonium chloride, and triclosan. As an exemplary embodiment, the present invention entails a method to directly measure the extent to which a potential antiviral substance inhibits the effects of viral infection in a tissue culture. Plaque reduction is recommended for virus and cell line combinations which produce a well-defined plaque. Other cytopathic effects (CPE) can be measured in those virus and cell lines which do not produce well-defined plaques, or where a specific cytopathic effect may be a defining characteristic.

To demonstrate plaque formation, Feline Infectious Peritonitis Type 2 (FIP Type 2) corona virus cultures were grown in CrFK cell culture. Untreated virus served as controls. Alternatively, virus suspensions were exposed to three concentrations of CPC for 300 seconds. The concentrations were produced by adding certified USP grade cetylpyridinium chloride powder to sterile water to produce 10 ml each of solution concentrations at 0.025%, 0.05%, and 0.10%. At the end of the exposure, the virus was titrated in CrFK cell culture. Viral titration is accomplished at each test article concentration by seeding 0.1 ml of a serial dilution of treated virus to the CrFK cell culture. Serial dilutions are prepared from 10⁻¹ to 10⁻⁸ of the original virus concentration, or five million/ml (6.7 log units) and each dilution then placed in each of four wells (replicates) of a 96 well microplate. The inoculated cells (80 μl per well) are then incubated at 37° C. in a CO₂ incubator for five days. They are then observed microscopically for CPE inhibition, reporting titers as TCID50/ml, also expressed as plaque forming units (pfu's). The corrected virus titer (# positives×dilution) is then reported in log/ml. Virucidal activity is thus defined as the number of logs reduction of virus titer between the test article and the control (Reed and Meunch). The following table demonstrates the efficacy of CPC in the destruction of FIP Type2 corona virus:

Bioassay of FIP-2 Corona Virus against CPC

| CPC % | FIP-2 Titer | Reduction in Titer | % Reduction |
|---|---|---|---|
| 0.10 | <3.50 logs/ml* | >3.20 logs | 99.97 |
| 0.05 | <3.50 logs/ml* | >3.20 logs | 99.97 |
| 0.025% | 3.30 logs/ml | 3.40 logs | 99.95 |
| Control | 6.70 logs/ml | N/A | N/A |

*Dilutions of 10⁻² and below at CPC concentrations of 0.10% and 0.05% did demonstrate cell toxicity and could not be read with certainty. No virus were observed.
Note:
6.7 log units = 5,000,000 virus particles; 3.30 log units = ˜2,100 virus particles.

In the present invention, a significant reduction in virus titer occurred as a result of a five minute exposure of a viral suspension containing five million virus/ml in the presence of a 0.025% concentration of CPC. This reduction transposes into a reduction of greater than 99.95% of viral particles present. Assuming the same killing effect on a surface area, the application of CPC at a concentration of 0.025%/in² would result in a greater than 3-log reduction of virus. Comparative testing with benzalkonium chloride and triclosan as substitute active ingredients in the three concentrations as employed with CPC produced disparate results and demonstrated increased cellular toxicity. With benzalkonium chloride, only the 0.10% exposure resulted in viral reduction of which a percentage reduction was approximately 20%. Triclosan demonstrated cellular toxicity at all three levels with no viral reduction at all three levels. In the present invention, CPC at a concentration of 0.025% in solution consistently demonstrated a greater than 3-log lethal toxicity of the FIP Type2 corona virus for five minutes without adverse cytopathic effects.

It would be recognized by those in the art that the FIP-2 corona virus represents an enduring and resilient viral specimen with high resistance to antibiotics and biocides. It is an enveloped virus, closely resembling the SARS virus, and is a surrogate for laboratory testing of the SARS virus. The corona viruses are hardy organisms and in the case of the SARS virus, has an ability to reside outside of host systems for extended periods of time. The virus has a resistance to drying, according to types, and in the case of SARS, may reside on surfaces for extended periods with full ability to transfer to a host and cause infection.

Based upon the studies of the present invention, application of a 0.025% CPC solution to solid surfaces via a spray device or wipe or direct liquid pour would result in an effective disinfectant application and eliminate the corona virus. Additionally, since the studies substantiate no adverse cellular affectation at the 0.025% solution level, the introduction of the certain compositions of the present invention to the buccal cavity and throat area in humans may provide a benign but effective antiseptic and inhibitor of the corona virus and other less hardy viruses. In addition, as the prior art will further support the antibiotic and antifungal properties associated with CPC, it would provide another benefit in the use of the compound for treatment of viral maladies such as the common cold or SARS. Since the corona virus is associated with the common cold and other rhinoviruses, the 0.025% solution to mucosal surfaces of the respiratory tract would benefit by then inhibiting secondary infections associated with bacteria.

1. A method for inactivating viral pathogens comprising:
(a) providing a virucidal composition comprising a liquid media containing less than 1% weight per volume cetylpyridinium chloride; and
(b) contacting the virucidal composition with a surface targeted for disinfection.

2. The method of claim 1, wherein the virucidal composition further comprises less than 0.5% weight per volume cetylpyridinium chloride.

3. The method of claim 1, wherein the virucidal composition further comprises less than 0.05% weight per volume cetylpyridinium chloride.

4. The method of claim 1, wherein the virucidal composition further comprises less than 0.03% weight per volume cetylpyridinium chloride.

5. The method of claim 1, wherein the virucidal composition further comprises at least one extender.

6. The method of claim 5, wherein the at least one extender is a cellulose ether.

7. The method of claim 6, wherein the cellulose ether is hydroxypropyl methylcellulose or sodium carboxymethylcellulose.

8. The method of claim 5, wherein the at least one extender is a non-ionic detergent.

9. The method of claim 8, wherein the non-ionic detergent is selected from the group consisting of: Tween 80, Triton, Tyloxapol, Pluronic and Span.

10. The method of claim 1, wherein the virucidal composition further comprises at least one enhancer.

11. The method of claim 10, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

12. The method of claim 1, wherein the liquid media is selected from the group consisting of: sterile water, potable water and sterile saline.

13. The method of claim 1, wherein the virucidal composition further comprises at least one enhancer and at least one extender.

14. The method of claim 13, wherein the at least one extender is a cellulose ether.

15. The method of claim 14, wherein the cellulose ether is hydroxypropyl methylcellulose or sodium carboxymethylcellulose.

16. The method of claim 13, wherein the at least one extender is a non-ionic detergent.

17. The method of claim 16, wherein the at least one extender is selected from a group consisting of: Tween 80, Triton, Tyloxapol, Pluronic and Span.

18. The method of claim 13, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

19. The method of 1, wherein the virucidal composition is contacted with the surface targeted for disinfection through the use of an applicator.

20. The method of 19, wherein the applicator is selected from the group consisting of: a wipe, a mop head and a sprayer.

21. The method of 1, wherein the virucidal composition further comprises a scented component.

22. The method of 1, wherein the virucidal composition is contacted to the surface for at least 3 minutes.

23. The method of 1, wherein the virucidal composition is contacted to the surface for at least 45 seconds.

24. A method for inactivating viral pathogens on inanimate surfaces comprising:
(a) providing a virucidal composition comprising a liquid media containing less than 1% weight per volume cetylpyridinium chloride; and
(b) contacting the virucidal composition with the targeted inanimate surface for disinfection.

25. The method of claim 24, wherein the virucidal composition further comprises less than 0.5% weight per volume cetylpyridinium chloride.

26. The method of claim 24, wherein the virucidal composition further comprises less than 0.05% weight per volume cetylpyridinium chloride.

27. The method of claim 24, wherein the virucidal composition further comprises less than 0.03% weight per volume cetylpyridinium chloride.

28. The method of claim 24, wherein the virucidal composition further comprises at least one extender.

29. The method of claim 28, wherein the at least one extender is a cellulose ether.

30. The method of claim 29, wherein the cellulose ether is hydroxypropyl methylcellulose or sodium carboxymethylcellulose.

31. The method of claim 28, wherein the at least one extender is a non-ionic detergent.

32. The method of claim 31, wherein the non-ionic detergent is selected from the group consisting of: Tween 80, Triton, Tyloxapol, Pluronic and Span.

33. The method of claim 24, wherein the virucidal composition further comprises at least one enhancer.

34. The method of claim 33, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

35. The method of claim 24, wherein the liquid media is an aqueous media.

36. The method of claim 24, wherein the liquid media is selected from the group consisting of: sterile water, potable water, sterile saline and fatty alcohols.

37. The method of claim 24, wherein the virucidal composition further comprises at least one enhancer and at least one extender.

38. The method of claim 37, wherein the at least one extender is a cellulose ether.

39. The method of claim 38, wherein the cellulose ether is hydroxypropyl methylcellulose or sodium carboxymethylcellulose.

40. The method of claim 37, wherein the at least one extender is a non-ionic detergent.

41. The method of claim 40, wherein the at least one extender is selected from a group consisting of: Tween 80, Triton, Tyloxapol, Pluronic and Span.

42. The method of claim 37, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

43. The method of 24, wherein the virucidal composition is contacted with the surface targeted for disinfection through the use of an applicator.

44. The method of 43, wherein the applicator is selected from the group consisting of: a wipe, a mop head and a sprayer.

45. The method of 24, wherein the virucidal composition further comprises a scented component.

46. The method of 24, wherein the virucidal composition is contacted to the surface for at least 3 minutes.

47. The method of 24, wherein the virucidal composition is contacted to the surface for at least 45 seconds.

48. A method for the treatment of virulent infections comprising:
(a) providing a pharmaceutically acceptable virucidal composition containing cetylpyridinium chloride in solution in the liquid media at a concentration of less than 0.05% weight per volume;
(b) topically applying the virucidal composition to an infected surface area of a host organism.

49. The method of claim 48, wherein the pharmaceutically acceptable virucidal composition further comprises less than 0.03% weight per volume cetylpyridinium chloride.

50. The method of claim 48, wherein the pharmaceutically acceptable virucidal composition further comprises less than 0.025% weight per volume cetylpyridinium chloride.

51. The method of claim 48, wherein the pharmaceutically acceptable virucidal composition further comprises at least one extender.

52. The method of claim 51, wherein the at least one extender is hydroxypropyl methylcellulose or sodium carboxymethylcellulose.

53. The method of claim 51, wherein the at least one extender is a non-ionic detergent.

54. The method of claim 53, wherein the non-ionic detergent is selected from the group consisting of: Tween 80, Triton, Tyloxapol, Pluronic and Span.

55. The method of claim 48, wherein the pharmaceutically acceptable virucidal composition further comprises at least one enhancer.

56. The method of claim 55, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate and zinc salicylate.

57. The method of claim 48, wherein the liquid media is an aqueous media.

58. The method of claim 48, wherein the liquid media is selected from the group consisting of: sterile water, potable water, sterile saline and fatty alcohols.

59. The method of claim 48, wherein the pharmaceutically acceptable virucidal composition further comprises at least one enhancer and at least one extender.

60. The method of claim 59, wherein the at least one extender is hydroxypropyl methylcellulose or sodium carboxymethylcellulose.

61. The method of claim 59, wherein the at least one extender is a non-ionic detergent.

62. The method of 61, wherein the non-ionic detergent is selected from the group consisting of: Tween 80, Triton, Tyloxapol, Pluronic and Span.

63. The method of claim 59, wherein the at least one extender is selected from the group consisting of: zinc oxide, zinc acetate, zinc bacitracin, zinc carbonate, zinc citrate, zinc iodate, zinc iodide, zinc peroxide, zinc propionate, zinc stearate and zinc salicylate.

64. The method of 48, wherein the pharmaceutically acceptable virucidal composition is contacted with the infected surface area in the form of an oral rinse.

65. The method of 48, wherein the pharmaceutically acceptable virucidal composition is contacted with the infected surface area in the form of a mist.

66. The method of 65, wherein the infected surface area is in the lungs of an organism.

67. The method of 48, wherein the pharmaceutically acceptable virucidal composition is contacted with the infected surface area in the form of a spray.

68. The method of 48, wherein the pharmaceutically acceptable virucidal composition further comprises a flavoring component.

69. The method of 48, wherein the pharmaceutically acceptable virucidal composition is contacted to the infected surface area for at least 3 minutes.

70. The method of 48, wherein the pharmaceutically acceptable virucidal composition is contacted to the infected surface area for at least 1 minute.

71. The method of 48, wherein the pharmaceutically acceptable virucidal composition is contacted to the infected surface area for at least 45 seconds.

72. The method of 48, wherein the infected surface area is a mucous membrane in the buccal cavity.

73. The method of 48, wherein the infected surface area is a mucous membrane in the nasal cavity.

74. The method of 48, wherein the infected surface area is in the oropharyngeal cavity.

75. A virucidal composition comprising:
(a) a liquid media;
(b) cetylpyridinium chloride in solution in the liquid media at a concentration of less than 1% weight per volume;
(c) an extender; and
(d) an enhancer.

76. The composition of claim 75, wherein the virucidal composition further comprises less than 0.5% weight per volume cetylpyridinium chloride.

77. The composition of claim 75, wherein the virucidal composition further comprises less than 0.05% weight per volume cetylpyridinium chloride.

78. The composition of claim 75, wherein the virucidal composition further comprises less than 0.03% weight per volume cetylpyridinium chloride.

79. The composition of claim 75, wherein the at least one extender is a cellulose ether.

80. The composition of claim 79, wherein the cellulose ether is hydroxypropyl methylcellulose or sodium carboxymethylcellulose.

81. The composition of claim 75, wherein the at least one extender is a non-ionic detergent.

82. The composition of claim 81, wherein the non-ionic detergent is selected from the group consisting of: Tween 80, Triton, Tyloxapol, Pluronic and Span.

83. The composition of claim 75, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

84. The composition of claim 75, wherein the liquid media is selected from the group consisting of: sterile water, potable water, sterile saline or fatty alcohols.

85. The composition of claim 75, wherein the composition is pharmaceutically acceptable.

86. A method for inactivating corona virus in a host organism comprising:
(a) providing a virucidal composition comprising a liquid media containing less than 0.05% cetylpyridinium chloride in solution; and
(b) contacting the virucidal composition with a surface contaminated with corona virus.

87. The method of claim 86, wherein the virucidal composition further comprises less than 0.03% weight per volume cetylpyridinium chloride.

88. The method of claim 86, wherein the virucidal composition further comprises less than 0.025% weight per volume cetylpyridinium chloride.

89. The method of claim 86, wherein the virucidal composition further comprises at least one extender.

90. The method of claim 89, wherein the at least one extender is hydroxypropyl methylcellulose or sodium carboxymethylcellulose.

91. The method of claim 89, wherein the at least one extender is a non-ionic detergent.

92. The method of claim 91, wherein the non-ionic detergent is selected from the group consisting of: Tween 80, Triton, Tyloxapol, Pluronic and Span.

93. The method of claim 86, wherein the virucidal composition further comprises at least one enhancer.

94. The method of claim 93, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate, zinc salicylate, cuprous iodide and cupric oleate.

95. The method of claim 86, wherein the liquid media is an aqueous media.

96. The method of claim 86, wherein the liquid media is selected from the group consisting of: sterile water, potable water, sterile saline and fatty alcohols.

97. The method of claim 86, wherein the virucidal composition further comprises at least one enhancer and at least one extender.

98. The method of claim 97, wherein the at least one extender is hydroxypropyl methylcellulose or sodium carboxymethylcellulose.

99. The method of claim 97, wherein the at least one extender is a non-ionic detergent.

100. The method of 99, wherein the non-ionic detergent is selected from the group consisting of: Tween 80, Triton, Tyloxapol, Pluronic and Span.

101. The method of claim 97, wherein the at least one extender is selected from the group consisting of: zinc oxide, zinc acetate, zinc bacitracin, zinc carbonate, zinc citrate, zinc iodate, zinc iodide, zinc peroxide, zinc propionate, zinc stearate and zinc salicylate.

102. The method of 86, wherein the virucidal composition is contacted with the surface contaminated with corona virus in the form of an oral rinse.

103. The method of 86, wherein the virucidal composition is contacted with the surface contaminated with corona virus in the form of a mist.

104. The method of 86, wherein the virucidal composition is contacted with the surface contaminated with corona virus in the form of a spray.

105. The method of 86, wherein the virucidal composition further comprises a flavoring component.

106. The method of 86, wherein the virucidal composition is contacted to the surface contaminated with corona virus for at least 3 minutes.

107. The method of 86, wherein the virucidal composition is contacted to the surface contaminated with corona virus for at least 1 minute.

108. The method of 86, wherein the virucidal composition is contacted to the surface contaminated with corona virus for at least 45 seconds.

109. The method of 86, wherein the surface contaminated with corona virus is a mucous membrane in the buccal cavity.

110. The method of 86, wherein the surface contaminated with corona virus is a mucous membrane in the nasal cavity.

111. The method of 86, wherein the surface contaminated with corona virus is in the oropharyngeal cavity.

112. A method for inactivating virus infections comprising:
(a) providing a virucidal composition comprising a liquid media containing less than 0.05% cetylpyridinium chloride in solution; and
(b) contacting the virucidal composition with a surface area of virally infected tissue.

113. The method of claim 112, wherein the virucidal composition further comprises less than 0.03% weight per volume cetylpyridinium chloride.

114. The method of claim 112, wherein the virucidal composition further comprises less than 0.025% weight per volume cetylpyridinium chloride.

115. The method of claim 112, wherein the virucidal composition further comprises at least one extender.

116. The method of claim 115, wherein the at least one extender is hydroxypropyl methylcellulose or sodium carboxymethylcellulose.

117. The method of claim 115, wherein the at least one extender is a non-ionic detergent.

118. The method of claim 117, wherein the non-ionic detergent is selected from the group consisting of: Tween 80, Triton, Tyloxapol, Pluronic and Span.

119. The method of claim 112, wherein the virucidal composition further comprises at least one enhancer.

120. The method of claim 119, wherein the at least one enhancer is selected from a group consisting of: zinc oxide, zinc acetate, zinc bacitracin, zinc carbonate, zinc citrate, zinc iodate, zinc iodide, zinc peroxide, zinc propionate, zinc stearate and zinc salicylate.

121. The method of claim 112, wherein the liquid media is an aqueous media.

122. The method of claim 112, wherein the liquid media is selected from the group consisting of: sterile water, potable water, sterile saline and fatty alcohols.

123. The method of claim 112, wherein the virucidal composition further comprises at least one enhancer and at least one extender.

124. The method of claim 123, wherein the at least one extender is hydroxypropyl methylcellulose or sodium carboxymethylcellulose.

125. The method of claim 123, wherein the at least one extender is a non-ionic detergent.

126. The method of 125, wherein the non-ionic detergent is selected from the group consisting of: Tween 80, Triton, Tyloxapol, Pluronic and Span.

127. The method of claim 123, wherein the at least one extender is selected from the group consisting of: zinc oxide, zinc acetate, zinc bacitracin, zinc carbonate, zinc citrate, zinc iodate, zinc iodide, zinc peroxide, zinc propionate, zinc stearate and zinc salicylate.

128. The method of 112, wherein the virucidal composition is contacted with the surface area of virally infected tissue in the form of an oral rinse.

129. The method of 112, wherein the virucidal composition is contacted with surface area of virally infected tissue in the form of a mist.

130. The method of 112, wherein the virucidal composition is contacted with the surface area of virally infected tissue in the form of a spray.

131. The method of 112, wherein the virucidal composition further comprises a flavoring component.

132. The method of 112, wherein the virucidal composition is contacted to the surface area of virally infected tissue for at least 3 minutes.

133. The method of 112, wherein the virucidal composition is contacted to the surface area of virally infected tissue for at least 1 minute.

134. The method of 112, wherein the virucidal composition is contacted to the surface area of virally infected tissue for at least 45 seconds.

135. The method of 112, wherein the surface area of virally infected tissue is a mucous membrane in the buccal cavity.

136. The method of 112, wherein the surface area of virally infected tissue is a mucous membrane in the nasal cavity.

137. The method of 112, wherein the surface area of virally infected tissue is in the oropharyngeal cavity.

138. The method of 112, wherein the surface area of virally infected tissue is in the lungs of an organism.
